(19) United States
(12) Patent Application Publication
(10) Pub. No.: US 2007/0071646 A1
(43) Pub. Date: Mar. 29, 2007

(54) System and method for regulating temperature inside an instrument housing

(76) Inventors: Alan E. Schoen, Saratoga, CA (US); Dennis M. Taylor, San Jose, CA (US); Jonathan W. Amy, West Lafayette, IN (US)

Correspondence Address: THERMO FINNIGAN LLC, 355 RIVER OAKS PARKWAY, SAN JOSE, CA, 95134, US

(21) Appl. No.: 11/241,714

(22) Filed: Sep. 29, 2005

(51) Int. Cl. G01N 33/48

(52) U.S. Cl. 422/68.1

(57) ABSTRACT

Embodiments of this invention regulate temperature inside an analytical instrument housing using a heat exchanger disposed adjacent an opening in the housing. Coolant is transferred to the heat exchanger to allow the heat exchanger to regulate a temperature of air drawn into the housing and over a temperature sensitive component. In certain embodiments, the coolant is also transferred to other structures and/or components in the instrument to regulate the temperatures of those structures and/or components.

### BACKGROUND OF THE INVENTION

1. Technical Field

The present invention relates to analytical instruments, and more particularly, to a system and method for regulating temperature inside an instrument housing.

2. Description of Related Art

Analytical instruments are apparatuses used to analyze material samples. Examples of analytical instruments include microscopes and spectrometers. A spectrometer disperses particles or radiation according to some property (e.g. mass or energy) and measures the dispersion. In a mass spectrometer, ions from a sample are dispersed according to mass-to-charge (m/z) ratios. The amount of dispersion is measured to determine properties of the sample. For example, this measurement may be used to identify a compound in the sample according to the mass of one or more elements in the compound. This measurement may also be used to determine the isotopic composition of one or more elements in the compound.

Analytical instruments such as microscopes and mass spectrometers often have temperature sensitive components. For example, analytical instruments may include passive components (e.g. resistors, capacitors or inductors) that output a different value depending on the passive component's operating temperature. For example, a resistor outputs a different resistance (e.g. in ohms) depending on the resistor's operating temperature. In addition there are strain gauge effects and other well known electronic quirks which are typically ignored as insignificant relative to the overall system performance. For example, the IC lead to circuit board connection at every pin has a thermocouple effect of c.a. 10 microvolts per degree Celsius.

Analytical instruments may also include active components (e.g. diodes or transistors) that exhibit a number of changes as a function of temperature. For example, bulk resistance of silicon decreases as temperatures rises, as does the forward voltage of a bipolar junction. Leakage currents also typically increase with an increase in operating temperature.

These temperature dependent changes may significantly affect an analytical instrument's operation. For example, the instrument's operation may rely on a digital logic circuit output signal. A typical digital logic circuit produces an output signal in delayed response to an input signal (e.g. a clock signal). The delay between input and output signals is often dependent on the temperature of an integrated circuit (IC) implementing the circuit because the switching speeds of the gates forming the IC is temperature dependent. In electrical engineering, a temperature dependent change in the output of a circuit component (e.g. the change in this delay) is sometimes referred to as “thermal drift”.

The term “thermal drift” is also sometimes used to refer to temperature induced deformation of materials. The degree of deformation is dependent on a particular material's coefficient of thermal expansion. For example, in a microscope with an arm supporting high magnification optics above a sample, the size of the arm and coefficient of thermal expansion of materials (such as aluminum) composing the arm sometimes cause a misalignment between consecutive microscope images. The deformation which causes the misalignment is sometimes referred to as “thermal drift” as well.

Therefore, as used herein, the term “thermal drift” will refer generally to changes to a component's characteristics (e.g. value, output or physical form) which are a function of the component's temperature. Short term thermal drifts are reversible changes in which a component's characteristic returns to its previous value once the temperature reverts to a previous value, however, even reversible changes do not return to their accurate initial conditions due to hysteresis effects. Permanent thermal drifts are changes which are not reversible and often depend on the component's operating temperature and therefore, in the context of circuits, on the circuit's thermal design.

In certain instruments, such as high precision spectrometers and high magnification microscopes, even minor thermal drift can have a significant impact on the instrument's accuracy and performance. To reduce the effect of thermal drift on an instrument's accuracy and performance, conventional methods typically attempt to control a thermal environment surrounding the entire instrument. For example, conventional methods often place an instrument having temperature sensitive components in a room having a precisely controlled climate. Under this method, the entire room is maintained within a specific temperature range. Individuals using the instrument work inside this climate-controlled environment, and therefore under potentially uncomfortable conditions.

Other methods to reduce the effect of thermal drift use software to mathematically adjust results. The software is calibrated and/or trained using empirical data, a process which may be expensive and time-consuming depending on the instrument and the precision desired.

### BRIEF SUMMARY OF THE INVENTION

The present invention provides apparatus and methods for regulating temperature inside an instrument housing. One method transfers coolant from a cooler to a heat exchanger and draws air through the heat exchanger into a housing enclosing a temperature sensitive component of the instrument.

Particular implementations of the method can include one or more of the following features. The coolant can be used to influence the temperature of another component of the instrument before returning the coolant to the cooler. The drawn air can be used to maintain each of a plurality of compartments at different substantially constant temperatures. The rate at which coolant is transferred to the heat exchanger can be based on data from a temperature sensor inside the housing or on the ambient temperature outside the housing.

The invention further includes a system that can perform such a method. Particular implementations of the system can include one or more of the following features. The instrument can include an analytical instrument such as mass spectrometer. The housing can comprise a plurality of compartments, each compartment maintained at a different substantially constant temperature. The instrument can include a temperature sensor inside the housing to measure the temperature of the air drawn over the temperature sensitive component. The instrument may comprise a dew point sensor.

The invention can be implemented to realize one or more of the following advantages. The thermal drift of temperature sensitive components can be reduced, thus reducing the effects of thermal drift on an instrument's accuracy and performance. The need for other methods such as software to mathematically compensate for the effects of thermal drift can be reduced or eliminated, these other methods typically being expensive and time-consuming, and dependent on the precision desired.

Other aspects of the invention will be apparent from the accompanying figures and the detailed description which follow.

### BRIEF DESCRIPTION OF THE DRAWINGS

FIG. 1 illustrates an analytical instrument in accordance with one embodiment of this invention.

FIG. 2A is a top view of a quadrupole mass spectrometry system in accordance with one embodiment of this invention.

FIG. 2B is a side view of the mass spectrometry system of FIG. 2A.

FIG. 3A is a top view of a time-of-flight (TOF) mass spectrometry system in accordance with one embodiment of this invention.

FIG. 3B is a side view of part of the time-of-flight (TOF) mass spectrometry system of FIG. 3A.

FIG. 4 is a flow chart of a process in accordance with one embodiment of this invention.

FIG. 5 is a diagram illustrating coolant flow in a mass spectrometer in accordance with one embodiment of this invention.

FIG. 6 is a diagram illustrating air flow in accordance with one embodiment of this invention.

### DETAILED DESCRIPTION

A system and method for regulating temperature inside an instrument housing is disclosed. Coolant is transferred to a heat exchanger disposed adjacent to an opening in the housing. Using the coolant, the heat exchanger regulates the temperature of air drawn through the heat exchanger and into the housing.

By regulating the temperature of air drawn into the housing, the operating temperatures of temperature sensitive components inside the housing are also regulated. This regulation can reduce thermal drift and increase the instrument's accuracy and performance. In certain embodiments of the invention, the operating temperature of other components in the instrument are also regulated by transferring coolant used by the heat exchanger to other structures and/or components of the instrument.

The following provides variations and examples of various aspects of embodiments of the invention. It will be appreciated that the following variations and examples are not intended to be exhaustive or to limit the invention to the precise forms disclosed. These variations and examples are to provide further understanding of embodiments of the present invention.

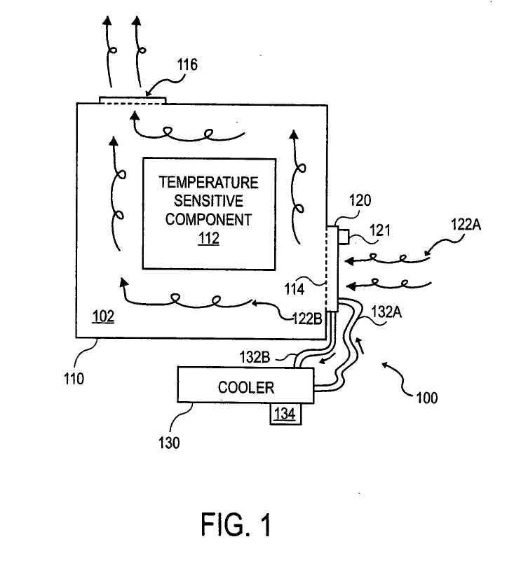

FIG. 1 illustrates an analytical instrument in accordance with one embodiment of this invention. In FIG. 1, system 100 includes an analytical instrument 102 having a temperature sensitive component 112 enclosed in a housing 110. The housing 110 has an opening 114 through which air enters the housing and an opening 116 through which air exits the housing. Heat exchanger 120 is disposed adjacent the opening 114. Heat exchanger 120 regulates the temperature of air drawn into the housing 110 and over the temperature sensitive component 112 using coolant transferred to the heat exchanger 120 from a cooler 130.

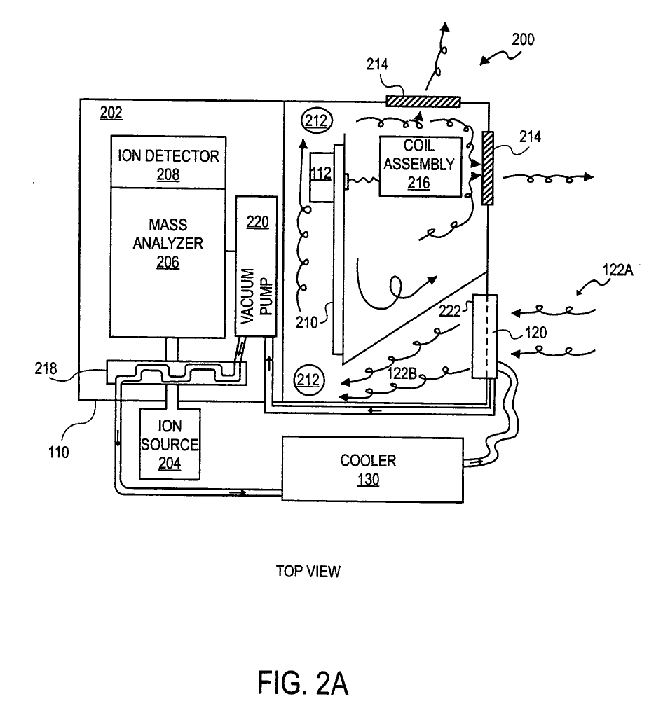

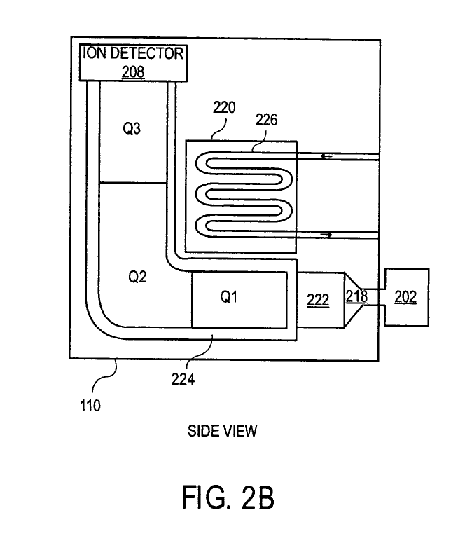

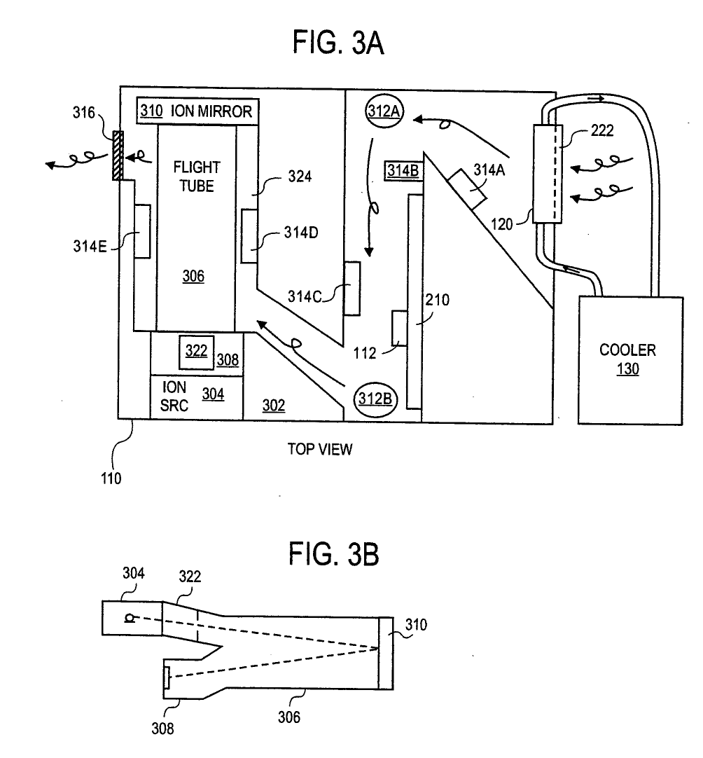

In one embodiment, analytical instrument 102 is a mass spectrometer. The mass spectrometer may be, for example, a quadrupole mass spectrometer (e.g. shown in FIG. 2A-2B), a time-of-flight (TOF) mass spectrometer (e.g. shown in FIG. 3), a magnetic sector mass spectrometer, an ion-trap mass spectrometer, a Fourier-transform mass spectrometer, an electrostatic mass spectrometer or a hybrid mass spectrometer.

The temperature sensitive component 112 may be one or a combination of an integrated circuit (IC), a digital-to-analog converter (DAC) subsystem, an operational amplifier, a gain setting resistor, a radio frequency (RF) detection diode, a trace on a circuit board, a tank circuit or a coil assembly, for example. In certain embodiments, the temperature sensitive component 112 is part of a control system, e.g. a circuit controlling the analytical instrument, as will be described in further detail below.

In FIG. 1, housing 110 has a single compartment through which air flows. In other embodiments, housing 110 includes a plurality of compartments (e.g. shown in FIG. 2A). Each of the plurality of compartments may contain a temperature sensitive component (e.g. an IC) whose temperature, and therefore thermal drift, is regulated by regulating the temperature of air in the compartment. Each of the plurality of compartments may additionally or alternatively contain a component that may otherwise benefit from temperature regulation (e.g. cooling), as will be in further detail described below.

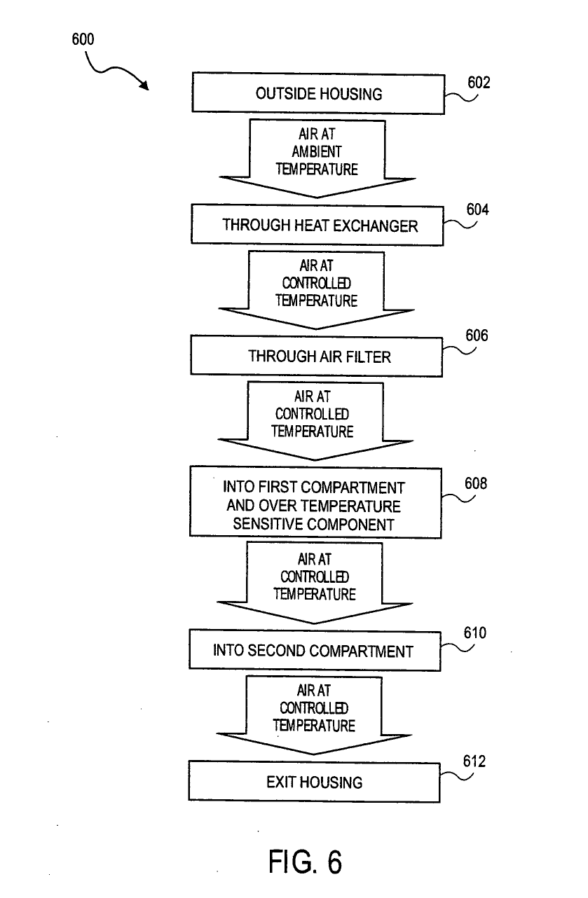

Openings 114 and 116 allow air to flow through housing 110. An air flow path in accordance with one embodiment of this invention is described in further detail in relation to FIG. 6. The air drawn into the housing is used to regulate the temperature of temperature sensitive component 112 and, in certain embodiments of the invention, other instrument components in the same compartment or a different compartment in housing 110.

In certain embodiments, housing 110 is composed of a thermally conducive material, e.g. aluminum or copper, to help dissipate thermal energy away from the instrument. In certain embodiments, housing 110 is composed of electrically conductive material, e.g. aluminum or copper, to act as an electrical ground for a circuit (e.g. in a control system) disposed inside the housing. In certain embodiments, housing 110 is composed of corrosion resistant material, e.g. titanium.

In certain embodiments, to protect against corrosion, housing 110 and heat exchanger 120 are composed of similar material. For example, in one embodiment, housing 110 and heat exchanger 120 are both composed of aluminum. When the housing and heat exchanger are composed of the same material, physical contact between the housing and the heat exchanger does not expose either to galvanic corrosion.

The amount of physical contact between the heat exchanger 120 and the housing 110 depends on the dimensions of the heat exchanger 120 relative to the opening 114 in the housing 110. If the dimensions of the opening are small relative to the dimensions of the heat exchanger, more of the heat exchanger's surface may be in physical contact with the housing. Dimensions and shapes of the opening and the heat exchanger vary depending on the embodiment of the invention. In one embodiment, the opening is circular and the heat exchanger has a circular cross-section that fits into or over the opening. In another embodiment, the opening is rectangular and the heat exchanger has rectangular cross-section that fits into or over the opening. In one embodiment, the rectangular opening is approximately 6 to 7 inches by 9 to 10 inches and a portion of the heat exchanger slides into the opening, extending approximately 2 inches deep into the housing.

Heat exchanger 120 is disposed adjacent to the opening 114 such that air outside the housing flows into the housing through the heat exchanger. In FIG. 1, heat exchanger 120 is disposed adjacent the opening 114 yet outside the housing 110. In other embodiments, the heat exchanger 120 is disposed adjacent the opening 114 but inside the housing 110 (e.g. as shown in FIG. 3). In one embodiment, heat exchanger 120 is a bar and plate design having fins laid between sheets of metal. In other embodiments, heat exchanger 120 is a plate and frame heat exchanger. In one embodiment, heat exchanger 120 includes ⅜ inch tube connections to attach to a cooler 130.

Cooler 130 pumps coolant to and through heat exchanger 120 to allow the heat exchanger 120 to regulate the temperature of air drawn into the housing (e.g. air 122B) and over a temperature sensitive component 112. Typically, the coolant acts as a heat sink, absorbing thermal energy from air that flows through the heat exchanger 120 into the housing 110. In certain instances, to maintain the air inside the housing at a stable temperature, the coolant may also act as a heat source, adding thermal energy to the air that flows through the heat exchanger and into the housing.

In certain embodiments, the coolant flow rate is a function of the volume of air being drawn into the housing, the temperature of the air outside the housing (e.g. air 122A), and/or a temperature desired for the air inside the housing (e.g. air 122B). For example, in one embodiment, the coolant flow rate is proportional to the volume of air being drawn into the housing (rate_{coolant flow} ∝ volume _{air drawn into housing}). In certain embodiments, air is drawn into the housing using fans disposed inside the housing (e.g. in FIG. 2A). In those embodiments, the coolant flow rate is also proportional to the speed of the fans (rate_{coolant flow} ∝ speed_{fan}). In certain embodiments, the volume of air drawn into the housing is proportional to the volume of open space in the housing. Therefore, among embodiments of the invention, the coolant flow rate may vary depending on the volume of open space in the housing.

In certain embodiments, the coolant flow rate is proportional to a difference between the temperature of the air outside the housing and a temperature desired for the air inside the housing (rate_{coolant flow} ∝ temperature_{air outside housing}—desired temperature_{air inside housing}). In other words, the coolant flow rate is greater when the difference is greater. Accordingly, the coolant flow rate may vary depending on the location of the instrument.

In certain embodiments, a temperature sensor 121 coupled to the heat exchanger provides data used to adjust the coolant flow rate. When the sensor is mounted outside the housing, data from the sensor may be used to adjust the coolant flow rate based on the temperature of air outside the housing. When the sensor is mounted inside the housing, data from the sensor may be used to adjust the coolant flow rate based on the temperature of air inside the housing. In the latter scenario, the sensor provides feedback data to allow the system 100 to determine when the temperature inside the housing reaches a desired temperature. Based on the feedback data, the system 100 may automatically increase or decrease the rate at which coolant is transferred to the heat exchanger, thereby allowing the heat exchanger to maintain the air inside the housing at a specific substantially constant temperature independent of temperatures outside the housing.

In one embodiment, the instrument is placed outdoors such that the air temperature outside the instrument's housing fluctuates with varying sunlight. A temperature sensor provides data to allow the system to adjust the coolant flow rate to maintain the air inside the housing at a substantially constant temperature independent of the air temperature outside the housing. During the daytime, the flow rate may be higher than at night, for example. In addition to the flow rate, in embodiments in which the heat exchanger substantially alters the air's temperature as it flows through the heat exchanger and into the housing, other variables such as the temperature of the coolant and/or the air flow rate may also be adjusted.

In certain embodiments, the heat exchanger alters the air's temperature only slightly to account for the minor fluctuations in ambient air temperature. These slight alterations still allow the heat exchanger to maintain the air temperature inside the housing stable, however. In such embodiments, the air temperature inside the housing is dependent on the ambient air temperature. For example, in one embodiment, the instrument is placed in a room with an ambient temperature of around 20° C.±2° C. The heat exchanger uses the coolant to maintain the air inside the instrument's housing at a stable 20° C. When the same instrument is moved to another room with an ambient temperature of around 15° C.±2° C., the heat exchanger again uses the coolant to maintain the air inside the instrument's housing stable, but this time at 15° C. rather than 20° C. Accordingly, the heat exchanger may maintain the air inside the housing at a temperature which is either dependent on or independent of the air outside the housing.

The coolant may be any liquid that can absorb thermal energy. For example, in certain embodiments, the coolant includes methanol, ethylene glycol, propylene glycol, water or nitrogen. In one embodiment, the coolant is a combination of propylene glycol and water, a 50/50 percentage mixture. In another embodiment, the coolant is a combination of ethylene glycol, water and a bitter-tasting agent (e.g. denatonium benzoate).

In FIG. 1, the coolant is transferred from the cooler 130 to the heat exchanger 120 via channels 132A-B. Channels 132A-B may be flexible or rigid. In certain embodiments, one or both channels 132A-B are tubes or pipes composed of TYGON® vinyl, polymerized vinyl chloride (PVC), chlorinated poly vinyl chloride (CPVC), or polypropylene. Channels 132A-B are connected to the cooler 130 and heat exchanger 120 using techniques which prevent the coolant from leaking at the connection junctions, e.g. using liquid-proof adhesives or connectors.

Cooler 130 pumps coolant through channels 132A-B. In one embodiment, cooler 130 is an immersion cooler, e.g. a Neslab cooler from Thermo Electron Corporation. Cooler 130 maintains the coolant at a certain temperature, e.g. 23° C. In one embodiment, cooler 130 is coupled to a dew point sensor 134. Data from dew point sensor 134 is used to determine the temperature at which to maintain the coolant in the cooler 130. By adjusting the coolant temperature based on data from the dew point sensor 134, the system 100 can prevent the heat exchanger 120 from cooling the air drawn into the housing to a temperature that would lead to condensation inside the housing. In one embodiment, dew point sensor 134 is coupled to a drip tray to collect condensation that may form near the system, e.g. near the cooler or near the heat exchanger.

In the embodiment of FIG. 1, the coolant flows from the cooler 130 to the heat exchanger 120 and back to the cooler 130. Air from outside the housing 110 flows through the heat exchanger 130, through an opening 114 in the housing, through a compartment containing a temperature sensitive component 112, and out an opening 116. More complicated coolant and air flows are discussed in the embodiments below.

FIG. 2A is a top view of a quadrupole mass spectrometry system in accordance with one embodiment of this invention. In FIG. 2A, the mass spectrometry system 200 includes a mass spectrometer 202 which includes an ion source 204, a mass analyzer 206 and an ion detector 208.

The ion source 204 ionizes a sample material under analysis (sometimes referred to as the analyte). In certain embodiments, the ion source is an atmospheric pressure chemical ionization (APCI) source or a heated electrospray ionization (ESI) source. In other embodiments, the ion source is an atmospheric pressure photo-ionization (APPI) source, an atmospheric pressure photo-chemical-ionization (APPCI) source, a matrix assisted laser desorption ionization (MALDI) source, an atmospheric pressure MALDI (AP-MALDI) source, an electron impact ionization (EI) source, a chemical ionization (CI) source, an electron capture ionization source, or a fast bombardment source or a secondary ions (SIMS) source. The ions from the ion source 204 are transported (e.g. by magnetic or electric fields) to the mass analyzer 206.

Mass analyzer 206 is coupled to ion source 204 and uses an electric or magnetic field to deflect the ions from the ion source 204. In a quadrupole mass spectrometer, the mass analyzer 206 includes a quadrupole (or “quad”) which consists of four parallel rods. In a triple quadrupole (or “triple quad”) mass spectrometer, three quadrupoles are used, such as Q1, Q2 and Q3 shown in FIG. 2B and described in further detail below. In other mass spectrometers, the mass analyzer is composed of other structures, e.g. a flight tube (in a time-of-flight mass spectrometer) or a ring electrode separating two hemispherical electrodes (in a 3D ion-trap mass spectrometer). In other embodiments, mass analyzer 206 is a linear ion trap mass analyzer, a Fourier Transform mass analyzer or an Orbitrap™ electrostatic mass analyzer manufactured by Thermo Electron Corporation (which employs the trapping of pulsed ion beams in an electrostatic quadro-logarithmic field created between an axial central electrode and a coaxial outer electrode).

Ion detector 208 is coupled to the mass analyzer 206 and detects ions exiting the mass analyzer 204. The ions detected produce a spectrum. Knowing properties of the mass spectrometer (e.g. the length of the mass analyzer and/or the strength of the magnetic or electric field used to accelerate the ions), the spectrum may be analyzed to determine numbers of ions at certain mass-to-charge ratios.

The mass spectrometer 202 of FIG. 2A includes a control system which includes one or more temperature sensitive components, e.g. temperature sensitive component 112 mounted on a printed circuit board (PCB) 210. The control system is coupled to the mass analyzer 206 to adjust operating properties of the mass analyzer. For example, in one embodiment, the control system adjusts magnetic or electric fields in the mass analyzer to alter trajectories of ions traveling through the mass analyzer. The control system may also receive data from ion detector 208. In certain embodiments, the control system may process the data before presenting it to a user.

Temperature sensitive component 112 may include, for example, an integrated circuit (IC), a digital-to-analog converter (DAC) subsystem, an operational amplifier, a gain setting resistor, a radio frequency (RF) detection diode, a trace, or a tank circuit mounted on the PCB. In other embodiments, temperature sensitive component 112 is inside PCB 210, e.g. a trace the inside the PCB.

In certain embodiments, temperature sensitive component 112 includes components coupled to the PCB but not mounted on or in the PCB. For example, in one embodiment, temperature sensitive component 112 may be or include coil assembly 216, which acts as a tank circuit for the control system. In FIG. 2A, coil assembly 216 is coupled to the PCB but is not disposed on or in the PCB.

Therefore, as can be understood from FIG. 2A, regulating the temperature of the air drawn into an instrument housing can regulate the temperature of more than one temperature sensitive component, e.g. temperature sensitive component 112 and coil assembly 216, simultaneously. Accordingly, thermal drift of more than one temperature sensitive component may be regulated simultaneously.

The embodiment shown in FIG. 2A includes fans 212 which drawn air into the housing. In certain embodiments, only one fan is used to drawn air into the housing. In other embodiments two, or more than two fans are used. In one embodiment, fans 212 are squirrel cage fans. In certain embodiment, the speed of the fan is dependent on the volume of open space the housing. In one embodiment, this speed is approximately 80 cubic feet/minute (CFM). Fans 212 draw ambient air 122A, through heat exchanger 120 and air filter 222, and into the housing 110.

Air filter 222 is disposed adjacent to the heat exchanger 120. Similar to heat exchanger 120, air filter 222 may be disposed inside or outside the housing. In the embodiment shown in FIG. 2A, the air filter is disposed inside the housing 110 while the heat exchanger 120 is disposed outside the housing 110. Therefore, in FIG. 2A, air outside the housing (e.g. air 122A) flows through the heat exchanger 120, through an opening in the housing (e.g. opening 114), through the air filter 222 and into the housing 110. The air then flows cross PCB 210, over temperature sensitive component 112, around coil assembly 216 and out one of the exit openings 214.

In other embodiments, the air filter is disposed outside the housing 110 while the heat exchanger 120 is disposed inside the housing 110, as shown in FIG. 3A. In such an embodiment, air outside the housing (e.g. 122A) flows through the air filter 222, through the opening, through the heat exchanger 120, and then into the housing 110. In other embodiments, the air filter 222 and heat exchanger 120 are both disposed outside the housing 110 or both disposed inside the housing 110.

Like heat exchanger 120, air filter 222 may be a variety of dimensions and shapes. For example, air filter 222 may be a round panel air filter with a radius that allows the filter to cover a circular opening. Air filter 222 may also be a rectangular panel air filter with a width and height that allows the filter to cover a rectangular opening.

In certain embodiments, after the coolant is used to maintain the drawn air at a substantially constant temperature, thereby minimizing thermal drift experienced by temperature sensitive components inside the housing, the coolant may be used for other purposes, e.g. to dissipate heat from a vacuum pump or a radio frequency (RF) generator.

FIG. 2A shows a vacuum pump 220 (e.g. a turbo pump), coupled to the mass analyzer. The vacuum pump 220 creates a vacuum inside the mass spectrometer. In the embodiment shown in FIG. 2A, coolant flows from the cooler 130 to the heat exchanger 120 and to the vacuum pump to regulate the temperature of the pump. In most embodiments, the coolant regulates the vacuum pump primarily by dissipating heat from the vacuum pump. In one embodiment, the coolant regulates the vacuum pump by maintaining the pump relatively isothermal.

FIG. 2B shows one structure through which the coolant may flow to regulate the vacuum pump 220. FIG. 2B is a side view of the mass spectrometry system of FIG. 2A. In FIG. 2B, channel 226 carries the coolant in an S-like path across and/or through the pump. In other embodiments, the channel may carry the coolant through a different shaped path. The channel 226 may be part of a structure disposed adjacent to the pump or may be inside the pump.

For example, the channel 226 may be part of a “water jacket” surrounding the vacuum pump. A water jacket is a liquid-filled (e.g. water-filled) void surrounding a device. Water jackets typically are metal (e.g. copper) sheaths having intake and outlet vents to allow liquids to be pumped through the void.

In certain embodiments, channel 226 is part of a water jacket surrounding a radio frequency (RF) generator or amplifier. In those embodiments, the coolant is used to dissipate heat originating from the RF generator or amplifier. The coolant may also be used to maintain the RF generator or amplifier relatively isothermal.

In FIG. 2A, after the coolant is used to regulate the vacuum pump, the coolant flows to structure 218 disposed between the ion source 204 and the mass analyzer 206. In one embodiment, the structure 218 is an aluminum or copper isothermal flange which buffers the mass analyzer 206 from one or more heat sources associated with the ion source 204. In one embodiment, the coolant flows through channels inside the structure 218. These channels may be formed using explosion techniques, for example. In other embodiments, the coolant flows through tubes or pipes mounted on the structure 218. In FIG. 2A, after the coolant flows through structure 218, the coolant returns to cooler 130.

Therefore, as can be understood from FIG. 2A and 2B, the coolant used by the heat exchanger to regulate the temperature of air drawn into the housing may also be used to regulate the temperature of the other structures or components. The coolant absorbs thermal energy from these structures or components, which can enable the instrument to function more efficiently. Alternatively, the coolant can impart thermal energy to these structures or components. Accordingly, as used herein, the phrase “regulating the temperature,” or similar phrases, encompasses maintaining the temperature relatively constant (or isothermal), as well as dissipating heat.

These other structures or components may be disposed inside or outside the instrument housing. For example, the coolant may regulate the thermal temperature of heating sources associated with ion source 204 which is disposed outside the housing 110 in FIG. 2A.

In certain embodiments, the other structure or component to be regulated is or includes a radio frequency (RF) amplifier, a vacuum pump, a flange disposed between an ion source and a mass analyzer, a quadrupole, or a flight tube. For example, in one embodiment, the coolant is used to cool a radio frequency (RF) amplifier. By cooling an RF amplifier using coolant rather than a fan, mechanical noise and vibrations in the system, which often lead to undesirable microphonics, is reduced. Microphonics is a phenomenon in which certain components in electronic devices transform mechanical vibrations into an undesired electrical signal (noise). Therefore, by using the coolant to cool the RF amplifiers, microphonics may be reduced and the instrument's performance improved.

In addition to using to the coolant to regulate the temperature of these other structures or components, fans may route the temperature regulated air inside the housing to various compartments enclosing these other structures or components. For example, a fan may be used to draw the air in the housing into a compartment (e.g. compartment 224 in FIG. 2B) to regulate a thermal environment surrounding a vacuum housing (not shown) containing one or more quadrupoles.

In FIG. 2B, compartment 224 encloses three quadrupoles, Q1, Q2 and Q3. Each quadrupole includes four parallel rods. Q1 and Q3 are hyperbolic quadrupoles. Q2 is a 90° square quadrupole collision cell which prevents the transmission of unwanted neutral species to the ion detector 208. Ions from ion source 202 flow past flange 218 through ion guide 222 and into Q1. Ion guide 222 accelerates and focuses the ions through an aperture (not shown) and into Q1. Ions having certain properties travel from Q1 to Q2, from Q2 to Q3 and then to ion detector 208.

The thermal environment surrounding a vacuum housing (not shown) containing Q1, Q2 and Q3 may be regulated by drawing the air inside the housing into the compartment before expelling the air back out of the housing. In certain embodiments, the temperature of compartment 224 may differ from the temperature of the compartment enclosing temperature sensitive component 112. In other words, in certain embodiments, the instrument housing may include a plurality of compartments and each of the plurality of compartments may be maintained at a different, yet substantially constant, temperature.

For example, one compartment may be maintained at 10° C. while another compartment may be maintained at 25° C. The temperature of each of these compartments may be maintained using air drawn through heat exchanger 120 into housing 110, the coolant, or a combination thereof.

As can be understood from the discussion above, in certain embodiments, coolant and/or air drawn into an instrument housing may be used to regulate the temperature of more than one component and/or more than one compartment in an analytical instrument. One such embodiment is shown in FIG. 3A.

FIG. 3A is a top view of a time-of-flight (TOF) mass spectrometry system in accordance with one embodiment of this invention. In FIG. 3A, temperature sensors 314A-E are mounted inside housing 110. Temperature sensors 314A-E may be or include a thermocouple, a temperature sensitive resistor (thermistor), a bi-metal thermometer, a resistance temperature detector (RTD) temperature sensor, or a silicon bandgap temperature sensor, for example.

In one embodiment, data from one or more of these sensors 314A-E are used to adjust the speed of fans 312A and 312B to alter the air flow rate into the housing 110, and thereby regulate the temperature of air inside the housing. In other embodiments, data from one or more of these sensors 314A-E are used to adjust the coolant flow rate or coolant temperature to regulate the temperature of air inside the housing.

In one embodiment, the data is transmitted to a control system inside the housing, e.g. a processor mounted on the printed circuit board 210. In other embodiments, the data is transmitted to a control system outside the housing, e.g. a processor inside or coupled to the cooler.

In FIG. 3A, fan 312A draws air through filter 222 and heat exchanger 120 into the housing 110. Air flows pass temperature sensor 314A-C and printed circuit board 210 having a temperature sensitive component 112. Fan 312B draws the air into a compartment 324 containing flight tube 306 (sometimes known as a drift tube). Flight tube 306 provides an electric field free region through which ions from ion source 304 may travel in a time-of-flight mass spectrometer.

Ion transit time through the flight tube 306 is dependent on the length of the flight tube. Changes in the thermal environment surrounding the flight tube may cause the length of the tube to change as the tube's material expands and contracts in response to changes in temperature. As can be seen from FIG. 3A, air drawn into the housing and through the heat exchanger is used to control the thermal environment surrounding the flight tube 306, therefore regulating the thermal drift of the flight tube. Temperature sensors 314D-E provide feedback to a control system to help maintain the thermal environment surrounding the flight tube 306 at a known temperature.

FIG. 3B is a side view of part of the time-of-flight (TOF) mass spectrometry system of FIG. 3A to help illustrate the ion flight path described above. Ions from ion source 304 are accelerated into the flight tube 306 by ion extractor or pusher 322. An ion mirror or reflectron 310 reflects the ions into an ion detector 308. The ion mirror or reflectron 310 compensates for the spread of kinetic energies of the ions as they enter the flight tube 306 and improves the resolution of the instrument 302. Because lighter ions have a higher velocity than heavier ions, the lighter ions reach the ion detector 308 sooner. In one embodiment, the output of the ion detector 308 is displayed on an oscilloscope (not shown) as a function of the time to produce the spectrum.

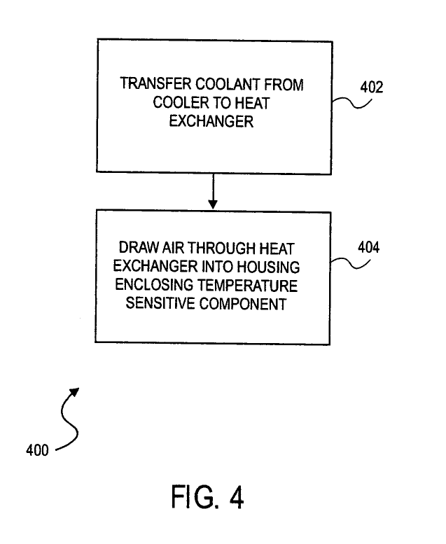

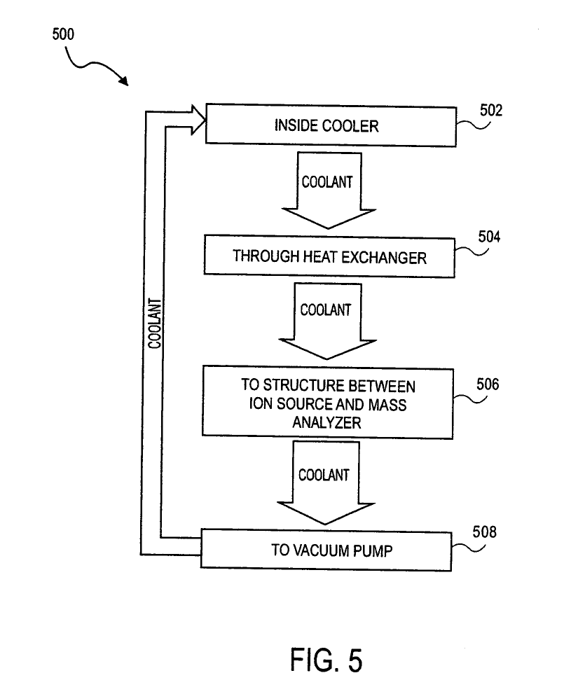

FIG. 4 is a flow chart of a process in accordance with one embodiment of this invention. At 402, coolant (e.g. ethylene glycol or propylene glycol) is transferred from a cooler to a heat exchanger. As previously discussed, the coolant may be transferred to other components (e.g. an isothermal flange) before or after the coolant is transferred to the heat exchanger. A coolant flow path in accordance with one embodiment of this invention is illustrated in FIG. 5.

At 404, air is drawn through the heat exchanger into a housing enclosing a temperature sensitive component (e.g. an IC). As previously discussed, this air may be drawn into the housing by a fan, such as squirrel cage fans mounted inside the housing. The air may also flow through a plurality of compartments. An air flow path in accordance with one embodiment of this invention is illustrated in FIG. 6.

FIG. 5 is a diagram illustrating coolant flow in a mass spectrometer in accordance with one embodiment of this invention. Coolant flow 500 starts at 502 inside a cooler (e.g. cooler 130). The cooler maintains the coolant at a substantially constant temperature (e.g. 23° C.). At 504, the coolant flows from the cooler through a heat exchanger (e.g. heat exchanger 120).

In the embodiment shown of FIG. 5, the coolant leaving the heat exchanger does not immediately return to the cooler but instead influences the temperature of other components of the analytical instrument before returning to the cooler. Specifically, at 506, the coolant flows through a structure disposed between an ion source and a mass analyzer. This structure may be, for example, flange 218 in FIG. 2A and 2B between ion source 204 and mass analyzer 206. In another embodiment, as described above, the coolant flows adjacent the structure rather than through it.

At 508, the coolant flows to a vacuum pump before returning to the cooler. In one embodiment, the coolant flows to the vacuum pump by flowing into a water jacket surrounding the pump, as previously described. In the embodiment shown in FIG. 5, unlike the embodiment of FIG. 2A, the coolant flows through a structure disposed between an ion source and a mass analyzer at 506 before flowing to a vacuum pump at 508. Accordingly, the coolant may flow to components and/or structures (including those not shown in FIG. 5, e.g. an RF amplifier) in any order and remain within the scope of this invention.

FIG. 6 is a diagram illustrating air flow in accordance with one embodiment of this invention. Air flow 600 starts outside the housing at 602.

At 604, the air (at ambient temperature) flows through a heat exchanger. Using coolant from a cooler, the heat exchanger regulates the temperature of the air drawn into the housing such that the air is at a controlled temperature. In one embodiment, the heat exchanger cools the air as it passes through the exchanger so that the temperature of the air inside the housing is lower than the temperature of the air outside the housing. In another embodiment, the heat exchanger may heat the air as it passes through the exchanger in order to maintain the temperature of the air inside the housing at a substantially constant temperature as the ambient air fluctuates around a certain temperature.

At 606, the air flows from the heat exchanger through an air filter. As previously discussed, in certain embodiments, the air may flow through the air filter before flowing through the heat exchanger. At 608, the air flows from the air filter into a first compartment and over a temperature sensitive component (e.g. temperature sensitive component 112). In one embodiment, the first compartment does not enclose the temperature sensitive component. Rather, the first compartment is an air intake region with an opening to another compartment enclosing the temperature sensitive component (e.g. in FIG. 2A and FIG. 3A). A fan (e.g. 212 or 312A) may be used to direct air in the intake region to the compartment enclosing the temperature sensitive component. In other embodiments, the first compartment encloses the temperature sensitive component (e.g. in FIG. 1). In certain embodiments (e.g. FIG. 1), the air flows from the first compartment out of the housing. In the first compartment containing active components, watts may be dissipated, which will perturb the temperature of the air, however, if the wattage is constant the perturbed temperature will be constant, provided the air flow is constant.

In the embodiment of FIG. 6, the air flows from the first compartment into a second compartment at 610. This second compartment may be, for example, compartment 224 in FIG. 2B which encloses a vacuum housing (not shown) containing one or more quadrupoles or compartment 324 in FIG. 3A which encloses a flight tube 306. The second compartment encloses the other structures or components of the instrument which may benefit from a thermally regulated environment, even if thermal drift of the structure or component may not affect the instrument's accuracy. In the second compartment containing active components, watts may be dissipated, which will perturb the temperature of the air, however, if the wattage is constant the perturbed temperature will be constant, provided the air flow is constant. At 612, the air exits the housing, e.g. via opening 116 in FIG. 1, openings 214 in FIG. 2A or opening 316 in FIG. 3A. In certain embodiments, these openings are covered by a vent or filter to prevent unwanted particles (e.g. dust) from entering the housing.

Thus, a system and method for regulating temperature inside an instrument housing is disclosed. In the above detailed description, numerous specific details are set forth in order to provide a thorough understanding of the present invention. However, it will be apparent to one of ordinary skill in the art that these specific details need not be used to practice the present invention. In other circumstances, well-known structures, materials, or processes have not been shown or described in detail in order not to unnecessarily obscure the present invention.

Although the present invention is described herein with reference to a specific preferred embodiment, many modifications and variations therein will readily occur to those with ordinary skill in the art. Accordingly, all such variations and modifications are included within the intended scope of the present invention as defined by the following claims.

Furthermore, the use of the phrase “one embodiment” throughout does not necessarily mean the same embodiment. Although these particular embodiments of the invention have been described, the invention should not be limited to these particular embodiments. Accordingly, the specification and drawings are to be regarded in an illustrative sense rather than a restrictive sense.

What is claimed is:

1. A system comprising:
an analytical instrument having a temperature sensitive component enclosed in a housing having an opening;
a heat exchanger, disposed adjacent to the opening, to regulate a temperature of air drawn into the housing and over the temperature sensitive component to maintain the drawn air at a substantially constant temperature independent of air temperature outside the housing; and
a cooler to transfer coolant to the heat exchanger.

2. The system of claim 1, wherein the analytical instrument is a mass spectrometer.

3. The system of claim 1, wherein the housing comprises a plurality of compartments and each compartment is maintained at a different substantially constant temperature using the drawn air, the coolant, or a combination thereof.

4. The system of claim 1, wherein the coolant is to flow adjacent to another component of the analytical instrument before returning to the cooler.

5. The system of claim 4, wherein the other component is selected from the group consisting of a radio frequency (RF) amplifier, a vacuum pump, a flange disposed between an ion source and a mass analyzer, a vacuum housing containing a quadrupole, a flight tube, or other temperature sensitive components.

6. The system of claim 1, further comprising a dew point sensor coupled to the cooler to determine a temperature for the coolant.

7. A mass spectrometry system comprising:
an ion source;
a mass analyzer coupled to the ion source;
an ion detector coupled to the mass analyzer;
a control system, coupled to the mass analyzer, having a temperature sensitive component enclosed in a housing having an opening;
a heat exchanger, disposed adjacent to the opening, to regulate a temperature of air drawn by a fan into the housing and over the temperature sensitive component to maintain the drawn air at a substantially constant temperature independent of air temperature outside the housing; and
a cooler, coupled to the heat exchanger, to transfer coolant to the heat exchanger.

8. The system of claim 7, further comprising a structure, disposed between the ion source and the mass analyzer, to receive the coolant flowing from the heat exchanger and to buffer thermally the mass analyzer from a heat source associated with the ion source.

9. The system of claim 7, wherein the mass analyzer comprises a quadrupole and the air drawn into the housing is further to regulate a thermal environment surrounding the quadrupole.

10. The system of claim 7, wherein the mass analyzer comprises a time-of-flight (TOF) flight tube and air drawn into the housing is further to regulate a thermal environment surrounding the flight tube.

11. The system of claim 7, further comprising a temperature sensor inside the housing to measure the temperature of the air drawn over the temperature sensitive component.

12. A method for regulating thermal drift in an analytical instrument comprising:
transferring coolant from a cooler to a heat exchanger; and
drawing air through the heat exchanger into a housing enclosing a temperature sensitive component of an analytical instrument, the heat exchanger maintaining the drawn air at a substantially constant temperature independent of air temperature outside the housing, minimizing temperature drift experienced by the temperature sensitive component.

13. The method of claim 12, further comprising using coolant leaving the heat exchanger to influence a temperature of another component of the analytical instrument before returning the coolant to the cooler.

14. The method of claim 12, further comprising using the drawn air, the coolant, or a combination thereof to maintain each of a plurality of compartments in the housing at different substantially constant temperatures.

15. The method of claim 12, wherein drawing air into the housing comprises of drawing the air at a rate dependent on a volume of space to be maintained at a substantially constant temperature.

16. A method for regulating thermal environments in a mass spectrometry system comprising:
transferring coolant from a cooler to a heat exchanger disposed adjacent to a first opening in a housing enclosing a control system controlling a mass spectrometer;
drawing air through the heat exchanger into an air intake compartment of the housing, the heat exchanger maintaining the drawn air at a substantially constant temperature independent of air temperature outside the housing;
drawing the air from the air intake compartment over a thermally sensitive component of the control system minimizing temperature drift experienced by the temperature sensitive component.; and
venting the air drawn over the thermally sensitive component out a second opening in the housing.

17. The method of claim 16, further comprising adjusting a rate at which the coolant is transferred to the heat exchanger based on data from a temperature sensor disposed inside the housing.

18. The method of claim 16, further comprising setting a temperature of the coolant based on ambient air temperature outside the housing.
